(19) United States
(12) Patent Application Publication
(10) Pub. No.: US 2022/0346705 A1
(43) Pub. Date: Nov. 3, 2022

(54) APPARATUS AND METHOD FOR SLEEP-DISORDERED BREATHING ESTIMATION BASED ON SOUND ANALYSIS

(71) Applicant: MaRI CO., LTD., Kyoto (JP)

(72) Inventors: Hirofumi TAKI, Kyoto (JP); Shigeaki OKUMURA, Kyoto (JP); Kazushi MORIMOTO, Kyoto (JP); Hiroaki OKINAKA, Kyoto (JP)

(73) Assignee: MaRI CO., LTD., Kyoto (JP)

(21) Appl. No.: 17/764,710

(22) PCT Filed: Sep. 30, 2020
(86) PCT No.: PCT/IB2020/000830

§ 371 Date: Mar. 29, 2022

Related U.S. Application Data
(60) Provisional application No. 62/910,408, filed on Oct. 3, 2019.
(60) Provisional application No. 62/908,545, filed on Sep. 30, 2019.

(51) Int. Cl. A61B 5/00; A61B 7/00; A61B 7/04; A61B 5/08; G16H 50/30

(52) U.S. Cl. CPC A61B 5/4818; A61B 7/003; A61B 7/04; A61B 5/7257; A61B 5/7221; A61B 5/7278; A61B 5/0816; G16H 50/30

(57) ABSTRACT

A snoring sound detection apparatus includes one or plural microphones that receive sounds produced by a subject, and a controller including circuitry which converts the sounds produced by the subject to received signals, converts the received signals to sound intensity signals, measures the periodicity of sound intensity signal using one or plural sound intensity signals, evaluates the validity of the periodicity of sound intensity signal in terms of respiratory rate, and detects snoring sound using the sound intensity and the validity of the periodicity of sound intensity signal in terms of respiratory rate.

### CROSS-REFERENCE TO RELATED APPLICATIONS

The present application is based upon and claims the benefits of priority to U.S. Provisional Application No. 62/908,545, filed Sep. 30, 2019, and U.S. Provisional Application No. 62/910,408, filed Oct. 3, 2019. The entire contents of all of the above applications are incorporated herein by reference.

### TECHNICAL FIELD

The present invention is directed to an apparatus and method that detect the sounds related to snoring and estimate sleep-disordered breathing and sleep apnea based on sound analysis.

### BACKGROUND ART

Obstructive sleep apnea is the most common form of sleep-disordered breathing (NPL 1). Obstructive sleep apnea is a sleep disorder in which breathing is repeatedly interrupted during sleep (NPL 2). Sleep apnea causes not only sleeplessness but also the increased incidence of various diseases and symptoms, e.g. high blood pressure, heart attack, cardiac arrhythmia, stroke and depression. Several inventors and researchers have focused on diagnosing obstructive sleep apnea using multi-parametric approach based on sound analysis (PL 1, NPL 3). However, most of the parameters employed by the multi-parametric approach are not suitable for the detection of snoring or sleep-disordered breathing. In addition, many of them are based on existing speech analysis technology, and in previous studies the frequency band lower than 3 Hz has been overlooked (NPL 4, NPL 5).

### CITATION LIST PATENT LITERATURE

PL 1 Udantha Abeyratne, Asela Samantha Karunajeewa, Houman Ghaemmaghami, “Multi-parametric analysis of snore sounds for the community screening of sleep apnea with non-gaussianity index, US20120004749A1.

### CITATION LIST NON PATENT LITERATURE

NPL 1 https://www.thoracic.org/patients/patient-resources/breathing-in-America/resources/chapter-23-sleep-disordered-breathing.pdf
NPL 2 https://www.sleepfoundation.org/sleep-apnea
NPL 3 Nir Ben-Israel, Ariel Tarasiuk, Yaniv Zigel, “Obstructive apnea hypopnea index estimation by analysis of nocturnal snoring signals in adults,” Sleep 2012 September; 35(9): 1299-1305.
NPL 4 Li-Ang Lee, Yu-Lun Lo, Jen-Fang Yu, Gui-She Lee, Yung-Lun Ni, Ning-Hung Chen, Tuan-Jen Fang, Chung-Guei Huang, Wen-Nuan Cheng, Hsueh-Yu Li, “Snoring sounds predict obstruction sites and surgical response in patients with obstructive sleep apnea hypopnea syndrome,” Sci Rep. 2016; 6: 30629.
NPL 5 Li-Ang Lee, Jen-Fang Yu, Yu-Lun Lo, Yen-Sheng Chen, Ding-Li Wang, Chih-Ming Cho, Yung-Lun Ni, Ning-Hung Chen, Tuan-Jen Fang, Chung-Guei Huang, Hsueh-Yu Li, “Energy types of snoring sounds in patients with obstructive sleep apnea syndrome: A preliminary observation,” PLoS One 2012; 7(12): e53481.

### SUMMARY OF THE INVENTION

According to an aspect of the present invention, a snoring sound detection apparatus includes one or plural microphones that receive sounds produced by a subject, and a controller including circuitry which converts sounds produced by the subject to received signals, converts the received signals to a plurality of sound intensity signals, measures the periodicity of sound intensity signal using one or plural sound intensity signals, evaluate the validity of the periodicity of sound intensity signal in terms of respiratory rate, and detect snoring sound using the sound intensity and the validity of the periodicity of sound intensity signal in terms of respiratory rate.

According to another aspect of the present invention, a snoring sound detection method includes acquiring sounds produced by a subject; converting the sounds produced by the subject to received signals, converting the received signals to sound intensity signals, measuring the periodicity of sound intensity signal using one or plural sound intensity signals, evaluating the validity of the periodicity of sound intensity signal in terms of respiratory rate, and detecting snoring sound using the sound intensity and the validity of the periodicity of sound intensity signal in terms of respiratory rate.

### BRIEF DESCRIPTION OF DRAWINGS

A more complete appreciation of the invention and many of the attendant advantages thereof will be readily obtained as the same becomes better understood by reference to the following detailed description when considered in connection with the accompanying drawings, wherein:

FIG. 1 is a schematic diagram of a sleep-disordered breathing estimation apparatus based on sound analysis, where snoring sound is detected using the evaluation of periodicity of sound intensity signals in terms of respiratory rate.

FIG. 2 is a schematic diagram of a sleep-disordered breathing estimation method based on sound analysis, where snoring sound is detected using the evaluation of periodicity of sound intensity signals in terms of respiratory rate.

FIG. 3 shows a schematic diagram of a method for sleep-disordered breathing estimation based on sound analysis, where Fourier transform is applied to sound intensity signals for the evaluation of periodicity of sound intensity signals in terms of respiratory rate.

FIG. 4 is a schematic diagram of an apparatus for snoring sound detection based on sound analysis.

FIG. 5 is a schematic diagram of a method for snoring sound detection based on sound analysis.

FIG. 6 is a schematic diagram of a method for snoring sound detection that employs a process storing indices in order to calculate indices.

FIG. 7 is a schematic diagram of a method for snoring sound detection employing the condition that snoring sound has the second harmonic.

FIG. 8 is a schematic diagram of a method for snoring sound detection that investigates whether low frequency components of received signals are dominant.

FIG. 9 is a schematic diagram of a method for snoring sound detection that employs autocorrelation.

### DESCRIPTION OF EMBODIMENTS

The embodiments will now be described with reference to the accompanying drawings, wherein like reference numerals designate corresponding or identical elements throughout the various drawings.

A sleep-disordered breathing estimation apparatus according to an embodiment of the present invention calculates an index in order to estimate the prevalence of sleep-disordered breathing. The apparatus is provided with one or plural microphones 104, one or plural reception circuits 106, sound intensity conversion filter 108, intensity periodicity measurement filter 110, intensity periodicity filter 112, snoring sound detection filter 114, and system controller 118 that controls the reception circuit 106, the sound intensity conversion filter 108, the intensity periodicity measurement filter 110, the intensity periodicity evaluation filter 112, and the snoring sound detection filter 114.

A sleep-disordered breathing estimation apparatus according to an embodiment of the present invention calculates an index in order to estimate the prevalence of sleep-disordered breathing. The apparatus is provided with one or plural microphones 104, one or plural reception circuits 106, sound intensity conversion filter 108, intensity periodicity measurement filter 110, intensity periodicity evaluation filter 112, snoring sound detection filter 114, sleep-disordered breathing estimation filter 116, and system controller 118 that controls the reception circuit 106, the sound intensity conversion filter 108, the intensity periodicity measurement filter 110, the intensity periodicity evaluation filter 112, the snoring sound detection filter 114, and the sleep-disordered breathing estimation filter 116.

The system controller 118 may be a computer that includes central processing unit (CPU) and a memory such as read-only memory (ROM) and random access memory (RAM). The CPU of the controller can be a single-core processor (which includes a single processing unit) or a multi-core processor. The computer may be a mobile device such as a personal digital assistant (PDA), laptop computer, field-programmable gate array, or cellular telephone.

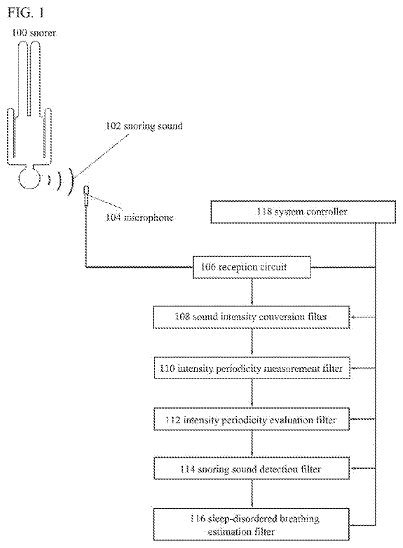

FIG. 1 shows a schematic diagram of an apparatus for sleep-disordered breathing estimation according to an embodiment of the present invention. One or plural microphones 104 with one or plural reception circuits 106 convert a plurality of sounds produced by a subject 100, including snoring sound 102, to a plurality of received signals. A microphone with a reception circuit in a cell phone is also applicable for the acquisition of plurality of received signals. A sound intensity conversion filter 108 converts a plurality of received signals to a plurality of sound intensity signals. An intensity periodicity measurement filter 110 measures the periodicity of sound intensity using one or plural sound intensity signals. An intensity periodicity evaluation filter 112 evaluates the validity of the periodicity of sound intensity in terms of respiratory rate. A snoring sound detection filter 114 detects snoring sound using the sound intensity and the validity of its periodicity acquired by an intensity periodicity evaluation filter 112. A sleep-disordered breathing estimation filter 116 calculates one or plural indices in order to estimate the prevalence of sleep-disordered breathing. The indices include the frequency of loud snore per hour, the total duration of loud snore per hour, and the total duration of sleeping per night, and the number of awakening per night.

A snoring sound detection method according to an embodiment of the present invention includes acquiring a plurality of sounds produced by a subject, converting the sounds produced by the subject to a plurality of received signals, converting the received signals to a plurality of sound intensity signals, measuring the periodicity of sound intensity signal using one or plural sound intensity signals, evaluating the validity of the periodicity of sound intensity signal in terms of respiratory rate, and detecting snoring sound using the sound intensity and the validity of the periodicity of sound intensity signal in terms of respiratory rate.

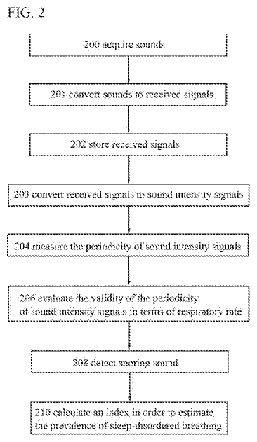

FIG. 2 shows a schematic diagram of a sleep-disordered breathing estimation method according to an embodiment of the present invention. In a process 200, the method acquires and stores sounds produced by a subject. In a process 201, the method converts sounds to received signals. In a process 202, the method stores received signals. The method may store filtered received signals and other information including acquired time and subject information. When the method stores filtered received signals, the method stores the filtered received signals after filter application to received signals. For example, when the method stores sound intensity signals, the storing process is located after the process 203. In the process 203, the method converts a plurality of received signals to a plurality of sound intensity signals. In a process 204, the method measures the periodicity of sound intensity using one or plural sound intensity signals. In a process 206, the method evaluates the validity of the periodicity of sound intensity in terms of respiratory rate. In a process 208, the method detects snoring sound using the sound intensity and the validity of its periodicity acquired by the process 206 that evaluate the validity of the periodicity of sound intensity. In a process 210, the method calculates one or plural indices in order to estimate the prevalence of sleep-disordered breathing.

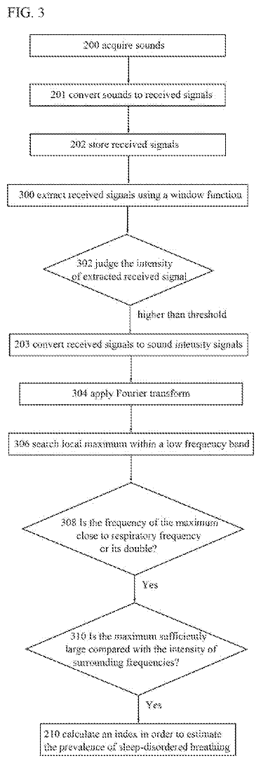

Fourier transform can be used in order to measure the periodicity of sound intensity signals in the process 204. FIG. 3 shows a schematic diagram of a method for sleep-disordered breathing estimation according to an embodiment of the present invention. In the process 200, the method acquires and stores sounds produced by a subject. In the process 201, the method converts sounds to received signals. In the process 202, the method stores received signals. In a process 300, the method extracts received signal using a window function, where the duration of the time window is 10 s or more. In a process 302, the method judges the intensity of extracted received signal. In the process 203, the method converts a plurality of received signals to a plurality of sound intensity signals. In a process 304, the method applies Fourier transform to extracted received signals. In a process 306, the method searches local maximums within a low frequency band. In a process 308, the method judges whether the frequency of the maximum of the local maximums is close to respiratory frequency or its double. In a process 310, the method judges whether the maximum of the local maximums is sufficiently large compared with the intensity of surrounding frequencies. The criteria of sufficiently large includes that the maximum of the local maximums is larger than three times the standard deviation of the local maximums. The threshold of two or 2.5 times of the standard deviation can be employed as the substitute of the threshold of three times. In the process 210, the method calculates one or plural indices in order to estimate the prevalence of sleep-disordered breathing. The period of intensity variation of the received signal caused by snoring sound is supposed to be closely related to respiratory frequency. Because the respiratory frequency of adults is from 0.2 to 0.3 Hz, the sampling interval in the frequency domain may be 0.1 Hz or less. Therefore, the duration of the time window for Fourier transform may be 10 s or more. In general, one of window functions, including rectangular window, B-spline window, Hann window, Hamming window, and Tukey window, is applied before Fourier transform.

For searching local maximum in the frequency domain in the process 306, the increase of the number of points in the frequency domain may be needed. Adding zeros to the beginning or/and end of each received signal, called zero padding, may be used before the process 304 in order to add more frequency points to the sound data in the frequency domain. Interpolation after the process 304 to the sound intensity data in the frequency domain also adds more frequency points to the sound intensity data in the frequency domain after Fourier transform, that is sound intensity data in the frequency domain.

In the process 304 Fourier-related transforms, including Wavelet transform, Laplace transform, fast Fourier transform, discrete Fourier transform, short-time Fourier transform, Z-transform and singular value decomposition, can be applicable as a substitute of Fourier transform.

In the process 203 signal envelope or its power can be employed in order to convert a plurality of received signals to a plurality of sound intensity signals. One of envelope estimation algorithms including rectification followed by low-pass filtering, magnitude of analytic signal, peak envelope and root-mean-square envelope, for the calculation of signal envelope may be used for the calculation of signal envelope. One of the metrics using rectification followed by low-pass filtering is defined by the following formula:

s_{RCT}(t)=F_{L}[ ]s(t)[ ], (1)

where F_{L}[ ] is a low-pass filter and s(t) is a received signal. One of low-pass filters is defined by the following formula in the frequency domain:

$${S}_{L}^{′} ( f ) = {S}^{′} ( f ) H ( f ) , \qquad ( 2 )$$

$$H ( f ) = \{ \begin{matrix}1 , & \vert f \vert \leq a \\ \frac{1}{2} \{ 1 + cos ( \frac{\vert f \vert - a}{b - a} π ) \} , & a < \vert f \vert \leq b \\ 0 , & b < \vert f \vert\end{matrix} \qquad ( 3 )$$

where f is frequency, a and b are positive numbers, and S′_{L}(f) is a signal in the frequency domain obtained by applying a low-pass filter to S′(f), a signal in the frequency domain. One of the metrics using magnitude of analytic signal is defined by the following formula:

S_{MA}(t)=|s_{A}(t)|, (4)

where S_{A}(t) is the analytic signal of a received signal. One of the metrics using peak envelope is defined by the following formula:

s_{P}(t)=max(|s(t±+kΔt)|)(−l≤k≤l), (5)

where max( ) is the maximum of the values, l is a natural number, k is an integer, and Δt is the sampling interval in the time domain. One of the metrics using root-mean-square envelope is defined by the following formula:

$${s}_{RMS} ( t ) = \sqrt{\frac{1}{2 l + 1} {\sum }_{k = - l}^{l} {\vert s ( t + k Δ t ) \vert}^{2}} . \qquad ( 6 )$$

In the process 203 moving average of the absolute value of received signal or its power can be used in order to convert a plurality of received signals to a plurality of sound intensity signals.

In the process 206 the periodicity of sound intensity is valid as snoring sound when the periodicity is close to respiratory frequency or its double, as shown in FIG. 3. The periodicity close to respiratory frequency indicates that snoring occurs at inhalation only. The periodicity close to twice respiratory frequency indicates that snoring occurs at both inhalation and exhalation.

In the process 206 the respiratory frequency may be adjusted according to subject information, because respiratory rate varies largely with subject, especially it depends on subject age.

As shown in FIG. 3, in the process 206 the method may judge the periodicity of sound intensity is valid when sound intensity signal in the frequency domain after Fourier transform application has one or more local maximums within the frequency band from 0.1 to 5 Hz, where the frequency of the maximum among local maximums within the frequency band from 0.1 to 5 Hz ranges from 0.15 to 2 Hz, because respiratory frequency and its double is supposed to range from 0.15 to 2 Hz.

In the process 210 the method may estimate apnea-hypopnea index using the sum of snoring duration per hour normalized by a certain duration that ranges from 20 to 40 s, because apnea-hypopnea index is the number of apnea and hypopnea per one hour of sleep, and mean apnea-hypopnea duration ranges from 20 to 40 s. Therefore, the method may set a snoring duration unit ranges from 20 to 40 s, the method may calculate the sum of snoring duration per one hour judged valid in a detecting snoring sound process, and the method may estimate apnea-hypopnea index using the sum of snoring duration per hour normalized by a snoring duration unit.

In the process 300 the duration of each extracted received signal may range from 20 to 40 s; and the method may estimate apnea-hypopnea index using the number of received signals per one hour judged valid in a detecting snoring sound process.

In the process 302 the method may exclude received signals of low intensity are excluded from analysis. The method may exclude received signals during a certain time after sleep and/or a certain time before waking from analysis. The method may exclude received signals during a subject is awake including speaking. The method may exclude received signals during a subject is supposed to have REM sleep.

The method may underestimate snoring duration when snoring continues for a certain period in the calculation of the sum of snoring duration, because simple snore continues a long time and simple snore does not mean hypopnea and apnea.

In the process 310 the method may evaluate the validity of the periodicity of sound intensity when the maximum among local maximums within the frequency band from 0.15 to 2 Hz is sufficiently large compared with the intensity of surrounding frequencies, because the dominance of snoring sound may cause large intensity in the frequency close to the respiratory frequency and its double. The order of the processes 308 and 310 may be switched.

A threshold for the maximum among local maximums within the frequency band from 0.15 to 2 Hz may be reduced when the frequency of the maximum is close to one or plural frequencies of the maximums of nearby received signals, because the frequency of the maximum caused by snoring sound is supposed to be close to that in nearby time. For example, the threshold that the maximum of the local maximums is larger than three times the standard deviation of the local maximums may be reduced to the threshold that the threshold that the maximum of the local maximums is larger than 2.5 times the standard deviation of the local maximums.

The method may increase the estimation value of apnea-hypopnea index when that frequency of the maximum among local maximums within the frequency band from 0.15 to 2 Hz varies largely in a sleep, because patients with severe sleep apnea syndrome have unstable respiratory frequency.

Snoring sound detection apparatus according to an embodiment of the present invention calculates an index in order to detect snoring. The apparatus is provided with one or plural microphones 104, one or plural reception circuits 106, fundamental frequency estimation filter 400, high-pass filter 402, envelope calculation filter 404, envelope periodicity estimation filter 406, envelope periodicity evaluation filter 408, snoring detection filter 410, and system controller 118 that controls the reception circuit 106, the fundamental frequency estimation filter 400, the high-pass filter application filter 402, the envelope calculation filter 404, the envelope periodicity estimation filter 406, the envelope periodicity evaluation filter 408, the snoring detection filter 410.

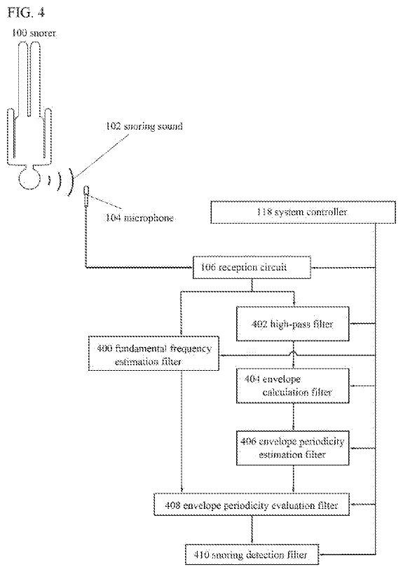

FIG. 4 shows a schematic diagram of an apparatus for snoring sound detection based on sound analysis. One or plural microphones 104 with one or plural reception circuits 106 convert a plurality of sounds produced by a subject to a plurality of received signals. A fundamental frequency estimation filter 400 estimates fundamental frequencies of received signals. A high-pass filter 402 applies a high-pass filter to received signals. An envelope calculation filter 404 calculates the envelopes of high-pass filtered received signals. An envelope periodicity estimation filter 406 estimates the periodicity of the envelopes of high-pass filtered received signals. An envelope periodicity evaluation filter 408 evaluates the periodicity of the envelopes of high-pass filtered received signals in terms of the fundamental frequencies of received signals. A snoring detection filter 410 calculates one or plural indices in order to detect snoring.

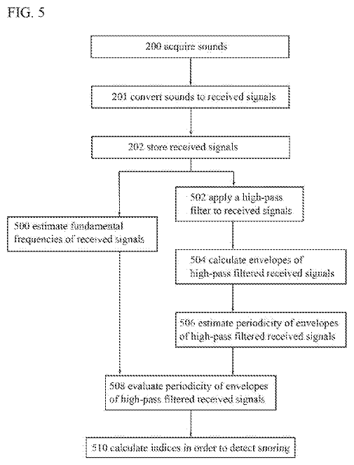

FIG. 5 shows a schematic diagram of a method for snoring sound detection based on sound analysis. In a process 200, the method acquires and stores sounds produced by a subject. In a process 201, the method converts sounds to received signals. In a process 202, the method stores received signals. The method may store filtered received signals and other information including acquired time and subject information. When the method stores filtered received signals, the method stores the filtered received signals after filter application to received signals. For example, when the method stores high-pass filtered received signals, the storing process is located after a process 502. In a process 500, the method estimates the fundamental frequencies of received signals. In the process 502, the method applies a high-pass filter to received signals. In a process 504, the method calculates the envelopes of high-pass filtered received signals. In a process 506, the method estimates the periodicity of the envelopes of high-pass filtered received signals. In a process 508, the method evaluates the periodicity of the envelopes of high-pass filtered received signals in terms of the fundamental frequencies of received signals, because the periodicity of the envelope of snoring sound after the elimination of the fundamental frequency of snoring sound using a high-pass filter, is supposed to be close to that of the fundamental frequency. In a process 510, the method calculates one or plural indices in order to detect snoring.

Fourier transform can be used in order to estimate the fundamental frequency of received signals in the process 500. The method may apply Fourier transform to received signals. The method may calculate intensity of received signals in the frequency domain. The method may search fundamental frequencies of received signals. Because snoring sound is one of time-varying signals, the fundamental frequency of snoring sound is supposed to be calculated using a short time window of 1 s or less. Therefore, the duration of the time window for Fourier transform is set to 1 s or less. One of window functions, including rectangular window, B-spline window, Hann window, Hamming window, and Tukey window, may be applied before Fourier transform.

In the process 500 addition of data without information to the beginning or/and end of each received signal, called zero padding, adds more frequency points to the sound data in the frequency domain. Interpolation to the intensity of received signals in the frequency domain after application of Fourier transform also adds more frequency points to the intensity of received signals in the frequency domain.

In the process 500 Fourier-related transforms, including Wavelet transform, Laplace transform, fast Fourier transform, discrete Fourier transform, short-time Fourier transform, Z-transform and singular value decomposition, can be applicable as a substitute of Fourier transform.

In the process 502 the method may apply a high-pass filter with a cutoff frequency to received signal, where the cutoff frequency of a high-pass filter is higher than the fundamental frequency of the received signal in order to eliminate the fundamental frequency of snoring sound.

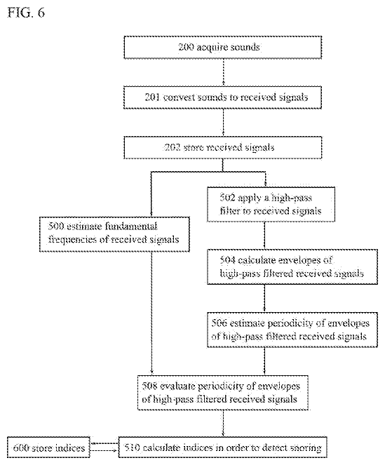

In a process 600 the method may store indices, where the method may use the stored indices for the calculation of one or plural indices in order to detect snoring in the process 510, as shown in FIG. 6.

In the process 500 the method may detect the local maxima of the intensity of received signal in the frequency domain, and the method may determine the local maximum with the lowest frequency as the fundamental frequency.

In the process 500 the method may determine the one of the local maxima of the intensity of received signal in the frequency domain as the fundamental frequency, where the factors used for the decision includes the amplitude of each local maximum, the intensity of each local maximum, the distance of each local maximum to other local maxima in the frequency domain, and the prominence of each local maximum.

In the process 500 the method may estimate the fundamental frequency of each received signal by calculating the periodicity of the signal amplitude in time domain.

In the process 500 the method may determine the fundamental frequency of received signals using a plurality of conditions including the fundamental frequencies are in the range from 10 to 300 Hz, because the fundamental frequency of snoring sound is supposed to be in the range from 10 to 300 Hz.

In the process 504 the method may calculate the envelope of high-pass filtered received signals under the condition of the envelope frequency being in the range from 10 to 300 Hz.

In the process 506 the method may apply Fourier transform to the envelopes of high-pass filtered received signals, and the method may search the maximum of local maximum of the intensity of each received signal in the frequency domain after Fourier transform in order to estimates periodicity of the envelopes of high-pass filtered received signals.

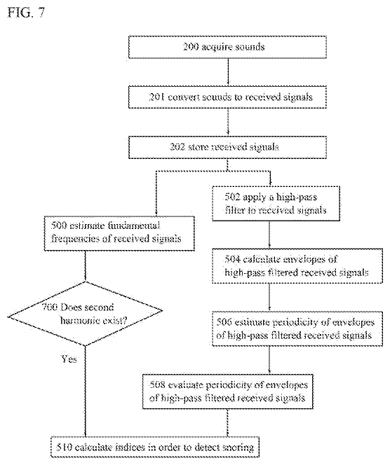

The method may employ the condition that snoring sound has the second harmonic. FIG. 7 shows a schematic diagram of a method for snoring sound detection employing the condition that snoring sound has the second harmonic. In the process 200, the method acquires and stores sounds produced by a subject. In the process 201, the method converts sounds to received signals. In the process 202, the method stores received signals. In the process 500, the method estimates the fundamental frequencies of received signals. In a process 700, the method searches second harmonics of received signals, because snoring sound is supposed to have second harmonics. In the process 502, the method applies a high-pass filter to received signals. In the process 504, the method calculates the envelopes of high-pass filtered received signals. In the process 506, the method estimates the periodicity of the envelopes of high-pass filtered received signals. In the process 508, the method evaluates the periodicity of the envelopes of high-pass filtered received signals in terms of the fundamental frequencies of received signals, because the periodicity of the envelope of snoring sound after the elimination of the fundamental frequency of snoring sound using a high-pass filter, is supposed to be close to that of the fundamental frequency. In the process 510, the method calculates one or plural indices in order to detect snoring.

In the process 510 the method may search the maximum of local maxima of the intensity of each received signal intensity in the frequency range from the fundamental frequency to the second harmonic of the received signal, where the method judges the received signal may include snoring sound when both the intensity of fundamental frequency and the intensity of the second harmonic of the received signal are higher than the maximum of local maxima of the intensity of each received signal in the frequency range from the fundamental frequency to the second harmonic of the received signal.

In the process 510 the method may search the maximum of the intensity of each received signal in the frequency range less than the fundamental frequency of the received signal, where the method judges the received signal may include snoring sound when the intensity of fundamental frequency of the received signal is larger than the maximum of the intensity of each received signal in the frequency range less than the fundamental frequency of the received signal.

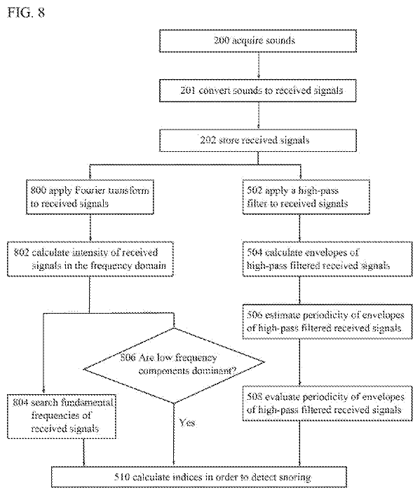

In a process 806 the method may investigate whether low frequency components of received signals are dominant, because low frequency components of received signals are supposed to be dominant. When the low frequency components are not dominant, the method judges that the received signal does not include snoring sound. FIG. 8 shows a schematic diagram of a method for snoring sound detection that investigates whether low frequency components of received signals are dominant. In the process 200, the method acquires and stores sounds produced by a subject. In the process 201, the method converts sounds to received signals. In the process 202, the method stores received signals. In a process 800 the method applies Fourier transform to received signals. In a process 802 the method calculates intensity of received signals in the frequency domain. In a process 804 the method searches the fundamental frequencies of received signals. In the process 806 the method investigates whether low frequency components of received signals are dominant. In the process 502, the method applies a high-pass filter to received signals. In the process 504, the method calculates the envelopes of high-pass filtered received signals. In the process 506, the method estimates the periodicity of the envelopes of high-pass filtered received signals. In the process 508, the method evaluates the periodicity of the envelopes of high-pass filtered received signals in terms of the fundamental frequencies of received signals, because the periodicity of the envelope of snoring sound after the elimination of the fundamental frequency of snoring sound using a high-pass filter, is supposed to be close to that of the fundamental frequency. In the process 510, the method calculates one or plural indices in order to detect snoring.

In the process 510 the method may judge the received signal may include snoring sound when the intensity of the fundamental frequency of received signal is dominant in the frequency domain.

In the process 510 the method may judge the received signal may include snoring sound when the intensity of the fundamental frequency of received signal accounts for 10% or more of the intensity sum of the received signal.

In the process 510 the method may judge the received signal may include snoring sound when the intensity and/or prominence of the fundamental frequency is higher than the intensity and prominence of other frequencies.

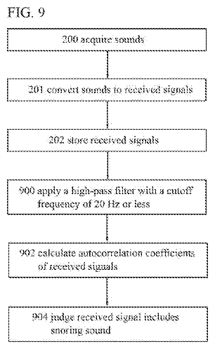

The method may employ autocorrelation. FIG. 9 shows a schematic diagram of a method for snoring sound detection that employs autocorrelation. In the process 200, the method acquires and stores sounds produced by a subject. In the process 201, the method converts sounds to received signals. In the process 202, the method stores received signals. In a process 900 the method applies a high-pass filter with a cutoff frequency of 20 Hz or less to received signals. In a process 902 the method calculates autocorrelation coefficients of received signals in the time domain using sliding windows of plural window widths, where the range of time lag for autocorrelation-coefficient calculation is included in the range from half the window width to twice the window width. In a process 904 the method judges received signal includes snoring sound when the autocorrelation coefficients of received signals become maximum in the case of employing a window width of 20 ms or more.

The method may store a plurality of received signals and/or filtered received signals. When the method stores filtered received signals, the method stores the filtered received signals after filter application to received signals.

### First Exemplary Embodiment

FIG. 1 shows a schematic diagram of an apparatus for sleep-disordered breathing estimation according to an embodiment of the present invention. One or plural microphones 104 with one or plural reception circuits 106 convert a plurality of sounds produced by a subject 100, including snoring sound 102, to a plurality of received signals. A microphone with a reception circuit in a cell phone is also applicable for the acquisition of plurality of received signals. A sound intensity conversion filter 108 converts a plurality of received signals to a plurality of sound intensity signals. An intensity periodicity measurement filter 110 measures the periodicity of sound intensity using one or plural sound intensity signals. An intensity periodicity evaluation filter 112 evaluates the validity of the periodicity of sound intensity in terms of respiratory rate. A snoring sound detection filter 114 detects snoring sound using the sound intensity and the validity of its periodicity acquired by an intensity periodicity evaluation filter 112. A sleep-disordered breathing estimation filter 116 calculates one or plural indices in order to estimate the prevalence of sleep-disordered breathing.

### Second Exemplary Embodiment

FIG. 2 shows a schematic diagram of a method for sleep-disordered breathing estimation according to an embodiment of the present invention. In the process 200, the method acquires and stores sounds produced by a subject. In the process 201, the method converts sounds to received signals. In the process 202, the method stores received signals. In the process 203, the method converts a plurality of received signals to a plurality of sound intensity signals. In the process 204, the method measures the periodicity of sound intensity using one or plural sound intensity signals. In the process 206, the method evaluates the validity of the periodicity of sound intensity in terms of respiratory rate. In the process 208, the method detects snoring sound using the sound intensity and the validity of its periodicity acquired by the process 206 that evaluate the validity of the periodicity of sound intensity. In the process 210, the method calculates one or plural indices in order to estimate the prevalence of sleep-disordered breathing.

### Third Exemplary Embodiment

FIG. 3 shows a schematic diagram of a method for sleep-disordered breathing estimation according to an embodiment of the present invention. In the process 200, the method acquires and stores sounds produced by a subject. In the process 201, the method converts sounds to received signals. In the process 202, the method stores received signals. In the process 300, the method extracts received signal using a window function, where the duration of the time window is 10 s or more. In the process 302, the method judges the intensity of extracted received signal. In the process 203, the method converts a plurality of received signals to a plurality of sound intensity signals by calculating the envelope of each received signal. In the process 304, the method applies Fourier transform to extracted received signals. In the process 306, the method searches local maximums within a low frequency band from 0.1 to 5 Hz. In the process 308, the method judges whether the frequency of the maximum of the local maximums is within a frequency band from 0.2 to 2 Hz. In the process 310, the method judges whether the maximum of the local maximums is sufficiently large compared with the intensity of surrounding frequencies. In the process 210, the method calculates one or plural indices in order to estimate the prevalence of sleep-disordered breathing.

### Forth Exemplary Embodiment

FIG. 4 shows a schematic diagram of an apparatus for snoring sound detection based on sound analysis. One or plural microphones 104 with one or plural reception circuits 106 convert a plurality of sounds produced by a subject to a plurality of received signals. A fundamental frequency estimation filter 400 estimates fundamental frequencies of received signals. A high-pass filter 402 applies a high-pass filter to received signals. An envelope calculation filter 404 calculates the envelopes of high-pass filtered received signals. An envelope periodicity estimation filter 406 estimates the periodicity of the envelopes of high-pass filtered received signals. An envelope periodicity evaluation filter 408 evaluates the periodicity of the envelopes of high-pass filtered received signals in terms of the fundamental frequencies of received signals. A snoring detection filter 410 calculates one or plural indices in order to detect snoring.

### Fifth Exemplary Embodiment

FIG. 5 shows a schematic diagram of a method for snoring sound detection based on sound analysis. In the process 200, the method acquires and stores sounds produced by a subject. In the process 201, the method converts sounds to received signals. In the process 202, the method stores received signals. In the process 500, the method estimates the fundamental frequencies of received signals. In the process 502, the method applies a high-pass filter to received signals. In the process 504, the method calculates the envelopes of high-pass filtered received signals. In the process 506, the method estimates the periodicity of the envelopes of high-pass filtered received signals. In the process 508, the method evaluates the periodicity of the envelopes of high-pass filtered received signals in terms of the fundamental frequencies of received signals, because the periodicity of the envelope of snoring sound after the elimination of the fundamental frequency of snoring sound using a high-pass filter, is supposed to be close to that of the fundamental frequency. In the process 510, the method calculates one or plural indices in order to detect snoring.

### Sixth Exemplary Embodiment

FIG. 6 shows a schematic diagram of a method for snoring sound detection that employs a process storing indices in order to calculate indices. In the process 200, the method acquires and stores sounds produced by a subject. In the process 201, the method converts sounds to received signals. In the process 202, the method stores received signals. In the process 500, the method estimates the fundamental frequencies of received signals. In the process 502, the method applies a high-pass filter to received signals. In the process 504, the method calculates the envelopes of high-pass filtered received signals. In the process 506, the method estimates the periodicity of the envelopes of high-pass filtered received signals. In the process 508, the method evaluates the periodicity of the envelopes of high-pass filtered received signals in terms of the fundamental frequencies of received signals, because the periodicity of the envelope of snoring sound after the elimination of the fundamental frequency of snoring sound using a high-pass filter, is supposed to be close to that of the fundamental frequency. In the process 510, the method calculates one or plural indices in order to detect snoring. In the process 600 the method may store indices, where the method may use the stored indices for the calculation of one or plural indices in order to detect snoring in the process 510.

### Seventh Exemplary Embodiment

FIG. 7 shows a schematic diagram of a method for snoring sound detection employing the condition that snoring sound has the second harmonic. In the process 200, the method acquires and stores sounds produced by a subject. In the process 201, the method converts sounds to received signals. In the process 202, the method stores received signals. In the process 500, the method estimates the fundamental frequencies of received signals. In the process 700, the method searches second harmonics of received signals, because snoring sound is supposed to have second harmonics. When the second harmonic does not exist, the method judges that the received signal does not include snoring sound. In the process 502, the method applies a high-pass filter to received signals. In the process 504, the method calculates the envelopes of high-pass filtered received signals. In the process 506, the method estimates the periodicity of the envelopes of high-pass filtered received signals. In the process 508, the method evaluates the periodicity of the envelopes of high-pass filtered received signals in terms of the fundamental frequencies of received signals, because the periodicity of the envelope of snoring sound after the elimination of the fundamental frequency of snoring sound using a high-pass filter, is supposed to be close to that of the fundamental frequency. In the process 510, the method calculates one or plural indices in order to detect snoring.

### Eighth Exemplary Embodiment

FIG. 8 shows a schematic diagram of a method for snoring sound detection that investigates whether low frequency components of received signals are dominant. In the process 200, the method acquires and stores sounds produced by a subject. In the process 201, the method converts sounds to received signals. In the process 202, the method stores received signals. In the process 800 the method applies Fourier transform to received signals. In the process 802 the method calculates intensity of received signals in the frequency domain. In the process 804 the method searches the fundamental frequencies of received signals. In the process 806 the method investigates whether low frequency components of received signals are dominant. In the process 502, the method applies a high-pass filter to received signals. In the process 504, the method calculates the envelopes of high-pass filtered received signals. In the process 506, the method estimates the periodicity of the envelopes of high-pass filtered received signals. In the process 508, the method evaluates the periodicity of the envelopes of high-pass filtered received signals in terms of the fundamental frequencies of received signals, because the periodicity of the envelope of snoring sound after the elimination of the fundamental frequency of snoring sound using a high-pass filter, is supposed to be close to that of the fundamental frequency. In the process 510, the method calculates one or plural indices in order to detect snoring.

The present invention has the following aspects.

1. A snoring sound detection apparatus, comprising: one or plural microphones that receive a plurality of sounds produced by a subject; and a controller comprising circuitry configured to convert a plurality of sounds produced by a subject to a plurality of received signals; convert a plurality of received signals to a plurality of sound intensity signals; measure the periodicity of sound intensity signal using one or plural sound intensity signals; evaluate the validity of the periodicity of sound intensity signal in terms of respiratory rate; and detect snoring sound using the sound intensity and the validity of the periodicity of sound intensity signal in terms of respiratory rate.

2. A sleep-disordered breathing estimation apparatus, comprising: one or plural microphones that receive a plurality of sounds produced by a subject; and a controller comprising circuitry configured to convert a plurality of sounds produced by a subject to a plurality of received signals; convert a plurality of received signals to a plurality of sound intensity signals; measure the periodicity of sound intensity signal using one or plural sound intensity signals; evaluate the validity of the periodicity of sound intensity signal in terms of respiratory rate; detect snoring sound using the sound intensity and the validity of the periodicity of sound intensity signal in terms of respiratory rate; and calculate one or plural indices in order to estimate the prevalence of sleep-disordered breathing.

3. A snoring sound detection method, comprising: acquiring a plurality of sounds produced by a subject; converting a plurality of sounds produced by a subject to a plurality of received signals; converting a plurality of received signals to a plurality of sound intensity signals; measuring the periodicity of sound intensity signal using one or plural sound intensity signals; evaluating the validity of the periodicity of sound intensity signal in terms of respiratory rate; and detecting snoring sound using the sound intensity and the validity of the periodicity of sound intensity signal in terms of respiratory rate.

4. A sleep-disordered breathing estimation method, comprising: acquiring a plurality of sounds produced by a subject; converting a plurality of sounds produced by a subject to a plurality of received signals; converting a plurality of received signals to a plurality of sound intensity signals; measuring the periodicity of sound intensity signal using one or plural sound intensity signals; evaluating the validity of the periodicity of sound intensity signal in terms of respiratory rate; detecting snoring sound using the sound intensity and the validity of the periodicity of sound intensity signal in terms of respiratory rate; and calculating one or plural indices in order to estimate the prevalence of sleep-disordered breathing.

5. A method for snoring sound detection or sleep-disordered breathing estimation according to 3 and 4, wherein the method comprises measuring the periodicity of sound intensity signal by Fourier transform; the duration of the time window for Fourier transform is 10 s or more, and one of window functions, including rectangular window, B-spline window, Hann window, Hamming window, and Tukey window, is employed as the window function of the time window for Fourier transform.

6. A method for snoring sound detection or sleep-disordered breathing estimation according to 5, further comprising: adding data without information to the beginning or/and end of each received signal.

7. A method for snoring sound detection or sleep-disordered breathing estimation according to 5, further comprising: applying interpolation to the sound intensity data in the frequency domain after Fourier transform.

8. A method for snoring sound detection or sleep-disordered breathing estimation according to 5, wherein the method comprises measuring the periodicity of sound intensity signal by one of Fourier-related transforms, including Wavelet transform, Laplace transform, fast Fourier transform, discrete Fourier transform, short-time Fourier transform, Z-transform and singular value decomposition, as a substitute of Fourier transform.

9. A method for snoring sound detection or sleep-disordered breathing estimation according to 3 and 4, wherein the method comprises converting a plurality of received signals to a plurality of envelopes of the received signals or to a plurality of a power of envelopes of the received signals, as a plurality of sound intensity signals.

10. A method for snoring sound detection or sleep-disordered breathing estimation according to 9, wherein the method comprises calculating a plurality of envelopes of the received signals by one of envelope estimation algorithm including rectification followed by low-pass filtering, magnitude of analytic signal, peak envelope and root-mean-square envelope.

11. A method for snoring sound detection or sleep-disordered breathing estimation according to 3 and 4, wherein the method comprises converting a plurality of received signals to a plurality of moving average of the absolute values of the received signals or to a plurality of a power of moving average of the absolute values of the received signals, as a plurality of sound intensity signals.

12. A method for snoring sound detection or sleep-disordered breathing estimation according to 3 and 4, wherein the method comprises evaluating the validity of the periodicity of sound intensity signal by the judgment criteria that a received signal is valid as snoring sound when the periodicity of the sound intensity of the received signal is close to respiratory frequency or its double.

13. A method for snoring sound detection or sleep-disordered breathing estimation according to 12, further comprising: adjusting the possible respiratory frequency according to subject information.

14. A method for snoring sound detection or sleep-disordered breathing estimation according to 5, wherein the method comprises evaluating the validity of the periodicity of sound intensity signal by the judgment criteria that a received signal is valid as snoring sound when both the following two conditions are satisfied; one is that sound intensity signal in the frequency domain after Fourier transform application has one or more local maximums within the frequency band from 0.1 to 5 Hz; the other is that frequency of the maximum among local maximums within the frequency band from 0.1 to 5 Hz ranges from 0.15 to 2 Hz.

15. A sleep-disordered breathing estimation method according to 4, wherein the method comprises estimating apnea-hypopnea index as an index that estimates the prevalence of sleep-disordered breathing; the apnea-hypopnea index is estimated using the sum of snoring duration per hour normalized by a snoring duration unit; the snoring duration unit ranges from 20 to 40 s; and the snoring duration is calculated by the duration of received signals detected as snoring sound.

16. A sleep-disordered breathing estimation method according to 4, wherein the method comprises estimating apnea-hypopnea index as an index that estimates the prevalence of sleep-disordered breathing; the validity of the periodicity of sound intensity signal in terms of respiratory rate is evaluated; the duration of sound intensity signal ranges from 20 to 40 s; and the apnea-hypopnea index is estimated using the number of sound intensity signals per one hour judged valid.

17. A method for snoring sound detection or sleep-disordered breathing estimation according to 3 and 4, wherein received signals of low intensity are excluded from analysis.

18. A method for snoring sound detection or sleep-disordered breathing estimation according to 3 and 4, further comprising: excluding received signals converted from sounds acquired during a certain time after sleep and/or a certain time before waking from analysis.

19. A method for snoring sound detection or sleep-disordered breathing estimation according to 3 and 4, further comprising: excluding received signals converted from sounds acquired during a subject is awake including speaking.

20. A method for snoring sound detection or sleep-disordered breathing estimation according to 3 and 4, further comprising: excluding received signals converted from sounds acquired during a subject is supposed to have REM sleep.

21. A sleep-disordered breathing estimation method according to 15, wherein the method comprises decreasing the sum of snoring duration when snoring continues for a certain period in the calculation of the sum of snoring duration.

22. A method for snoring sound detection or sleep-disordered breathing estimation according to 14, further comprising: evaluating the validity of the periodicity of sound intensity signal by the judgment criteria that a received signal is valid as snoring sound when the maximum among local maximums within the frequency band from 0.15 to 2 Hz is sufficiently large compared with the intensity of surrounding frequencies.

23. A method for snoring sound detection or sleep-disordered breathing estimation according to 22, further comprising: evaluating the validity of the periodicity of sound intensity signal by the judgment criteria that a received signal is valid as snoring sound when the maximum among local maximums within the frequency band from 0.15 to 2 Hz is large compared with the intensity of surrounding frequencies and the frequency of the maximum is close to one or plural frequencies of the maximums of nearby received signals.

24. A sleep-disordered breathing estimation method according to 14, wherein the method increases the estimation value of apnea-hypopnea index when the frequency of the maximum among local maximums within the frequency band from 0.15 to 2 Hz varies largely in a sleep.

25. A snoring sound detection apparatus, comprising: one or plural microphones that receive a plurality of sounds produced by a subject; and a controller comprising circuitry configured to convert a plurality of sounds produced by a subject to a plurality of received signals: estimate fundamental frequency of each received signal; apply a high-pass filter to received signals; calculate the envelopes of high-pass filtered received signals; evaluate the periodicity of the envelopes of high-pass filtered received signals in terms of the fundamental frequencies of received signals; and calculate one or plural indices in order to detect snoring.

26. A snoring sound detection method, comprising: acquiring a plurality of sounds produced by a subject; converting a plurality of sounds produced by a subject to a plurality of received signals; storing a plurality of received signals and/or filtered received signals; estimating the fundamental frequencies of received signals; applying a high-pass filter to received signals; calculating the envelopes of high-pass filtered received signals estimating the periodicity of the envelopes of high-pass filtered received signals; evaluating the periodicity of the envelopes of high-pass filtered received signals in terms of the fundamental frequencies of received signals; and calculating one or plural indices in order to detect snoring.

27. A snoring sound detection method according to 26, wherein the method comprises applying Fourier transform to received signals; calculating intensity of received signals in the frequency domain; and searching fundamental frequencies of received signals; the duration of the time window for Fourier transform is 1 s or less, and one of window functions, including rectangular window, B-spline window, Hann window, Hamming window, and Tukey window, is applied to received signals before Fourier transform.

28. A snoring sound detection method according to 27, further comprising: adding data without information to the beginning or/and end of each received signal.

29. A snoring sound detection method according to 27, further comprising: applying interpolation to intensity of received signals in the frequency domain after application of Fourier transform.

30. A snoring sound detection method according to 27, wherein the method comprises to employ one of Fourier-related transforms, including Wavelet transform, Laplace transform, fast Fourier transform, discrete Fourier transform, short-time Fourier transform, Z-transform and singular value decomposition, as a substitute of Fourier transform.

31. A snoring sound detection method according to 26, wherein the method comprises to apply a high-pass filter with a cutoff frequency to received signal; the cutoff frequency of a high-pass filter is higher than the fundamental frequency of the received signal.

32. A snoring sound detection method according to 26, further comprising: storing indices; using the stored indices for the calculation of one or plural indices in order to detect snoring.

33. A snoring sound detection method according to 26, wherein the method comprises detecting the local maxima of the intensity of received signal in the frequency domain; and determining the local maximum with the lowest frequency as the fundamental frequency.

34. A snoring sound detection method according to 33, wherein the method comprises determining one of the local maxima of the intensity of received signal in the frequency domain as the fundamental frequency by judging criteria using the amplitude of each local maximum, the intensity of each local maximum, the distance of each local maximum to other local maxima in the frequency domain, and the prominence of each local maximum.

35. A snoring sound detection method according to 26, wherein the method comprises estimating the fundamental frequency of each received signal by calculating the periodicity of the signal amplitude in time domain.

36. A method for snoring sound detection according to 26, wherein the method comprises determining the fundamental frequency of received signals using a plurality of conditions including the fundamental frequencies are in the range from 10 to 300 Hz.

37. A method for snoring sound detection according to 26, wherein the method comprises calculating the envelope of high-pass filtered received signals under the condition of the envelope frequency being in the range from 10 to 300 Hz.

38. A snoring sound detection method according to 26, wherein the method comprises applying Fourier transform to the envelopes of high-pass filtered received signals; and searching the maximum of local maximum of the intensity of each received signal in the frequency domain after Fourier transform in order to estimates periodicity of the envelopes of high-pass filtered received signals.

39. A snoring sound detection method, comprising: acquiring a plurality of sounds produced by a subject; converting a plurality of sounds produced by a subject to a plurality of received signals; storing a plurality of received signals and/or filtered received signals; estimating the fundamental frequencies of received signals; searching the second harmonics of received signals, applying a high-pass filter to received signals; calculating the envelopes of high-pass filtered received signals estimating the periodicity of the envelopes of high-pass filtered received signals; evaluating the periodicity of the envelopes of high-pass filtered received signals in terms of the fundamental frequencies of received signals; and calculating one or plural indices in order to detect snoring.

40. A snoring sound detection method according to 39, wherein the method comprises searching the maximum of local maxima of the intensity of each received signal in the frequency range from the fundamental frequency to the second harmonic of the received signal; and judging that the received signal may include snoring sound when both the intensity of fundamental frequency and the intensity of the second harmonic of the received signal are higher than the maximum of local maxima of the intensity of each received signal in the frequency range from the fundamental frequency to the second harmonic of the received signal.

41. A snoring sound detection method according to 39, wherein the method comprises searching the maximum of the intensity of each received signal in the frequency range less than the fundamental frequency of the received signal; and judging that the received signal may include snoring sound when the intensity of fundamental frequency of the received signal is larger than the maximum of the intensity of each received signal in the frequency range less than the fundamental frequency of the received signal.

42. A snoring sound detection method, comprising: acquiring a plurality of sounds produced by a subject; converting a plurality of sounds produced by a subject to a plurality of received signals; storing a plurality of received signals and/or filtered received signals; applying Fourier transform to received signals; calculating intensity of received signals in the frequency domain; searching the fundamental frequencies of received signals; investigating whether low frequency components of received signals are dominant; applying a high-pass filter to received signals; calculating the envelopes of high-pass filtered received signals estimating the periodicity of the envelopes of high-pass filtered received signals; evaluating the periodicity of the envelopes of high-pass filtered received signals in terms of the fundamental frequencies of received signals; and calculating one or plural indices in order to detect snoring.

43. A snoring sound detection method according to 42, wherein the method comprises judging that the received signal may include snoring sound when the intensity of the fundamental frequency of received signal is dominant in the frequency domain.

44. A method for snoring sound detection according to 42, wherein the method comprises judging that the received signal may include snoring sound when the intensity of the fundamental frequency of received signal accounts for 10% or more of the intensity sum of the received signal.

45. A method for snoring sound detection according to 42, wherein the method comprises judging that the received signal may include snoring sound when the intensity and/or prominence of the fundamental frequency is higher than the intensity and prominence of other frequencies.

46. A snoring sound detection method, comprising: acquiring a plurality of sounds produced by a subject; converting a plurality of sounds produced by a subject to a plurality of received signals; storing a plurality of received signals and/or filtered received signals, applying a high-pass filter with a cutoff frequency of 20 Hz or less to received signals; calculating autocorrelation coefficients of received signals in the time domain using sliding windows of plural window widths; and judging that received signal includes snoring sound when the autocorrelation coefficients of received signals become maximum in the case of employing a window width of 20 ms or more: the range of time lag for autocorrelation-coefficient calculation is included in the range from half the window width to twice the window width.

47. A method for snoring sound detection or sleep-disordered breathing estimation according to 3 and 4, further comprising: storing a plurality of received signals and/or filtered received signals.

Obviously, numerous modifications and variations of the present invention are possible in light of the above teachings. It is therefore to be understood that within the scope of the appended claims, the invention may be practiced otherwise than as specifically described herein.

### REFERENCE SIGNS LIST

100 snorer
102 snoring sound
104 microphone
106 reception circuit
108 sound intensity conversion filter
110 intensity periodicity measurement filter
112 intensity periodicity evaluation filter
114 snoring sound detection filter
116 sleep-disordered breathing estimation filter
118 system controller
200 acquire sounds
201 convert sound to received signals
202 store received signals
203 process
204 process
206 process
208 process
210 process
300 process
302 process
304 process
306 process
308 process
310 process
400 fundamental frequency estimation filter
402 high-pass filter
404 envelope calculation filter
406 envelope periodicity estimation filter
408 envelope periodicity evaluation filter
410 snoring detection filter
500 process
502 process
504 process
506 process
508 process
510 process
600 process
700 process
800 process
802 process
804 process
806 process
900 process
902 process
904 process

What is claimed is:

1. A snoring sound detection apparatus, comprising:
one or plural microphones that receive a plurality of sounds produced by a subject; and
a controller comprising circuitry configured to convert the sounds produced by the subject to a plurality of received signals, convert the received signals to a plurality of sound intensity signals, measure periodicity of sound intensity signal using one or plural sound intensity signals, evaluate validity of the periodicity of sound intensity signal in terms of respiratory rate, and detect snoring sound using the sound intensity and the validity of the periodicity of sound intensity signal in terms of respiratory rate.

2. A sleep-disordered breathing estimation apparatus, comprising:
one or plural microphones that receive a plurality of sounds produced by a subject; and
a controller comprising circuitry configured to convert the sounds produced by the subject to a plurality of received signals; convert the received signals to a plurality of sound intensity signals, measure periodicity of sound intensity signal using one or plural sound intensity signals, evaluate validity of the periodicity of sound intensity signal in terms of respiratory rate, detect snoring sound using the sound intensity and the validity of the periodicity of sound intensity signal in terms of respiratory rate, and calculate one or plural indices in order to estimate the prevalence of sleep-disordered breathing.

3. A snoring sound detection method, comprising:
acquiring a plurality of sounds produced by a subject;
converting the sounds produced by the subject to a plurality of received signals;
converting the received signals to a plurality of sound intensity signals;
measuring periodicity of sound intensity signal using one or plural sound intensity signals;
evaluating validity of the periodicity of sound intensity signal in terms of respiratory rate; and
detecting snoring sound using the sound intensity and the validity of the periodicity of sound intensity signal in terms of respiratory rate.

4. A sleep-disordered breathing estimation method, comprising:
detecting a snoring sound by the snoring sound detection method of claim 3; and
calculating one or plural indices to estimate prevalence of sleep-disordered breathing.

5. A snoring sound detection method according to claim 3, wherein the method comprises measuring the periodicity of sound intensity signal by Fourier transform;
the duration of the time window for Fourier transform is 10 s or more, and
one of window functions, including rectangular window, B-spline window, Hann window, Hamming window, and Tukey window, is employed as the window function of the time window for Fourier transform.

6. A snoring sound detection method according to claim 5, further comprising:
adding data without information to the beginning or/and end of each received signal.

7. A snoring sound detection method according to claim 5, further comprising:
applying interpolation to the sound intensity data in the frequency domain after Fourier transform.

8. A snoring sound detection method according to claim 5, wherein the method comprises measuring the periodicity of sound intensity signal by one of Fourier-related transforms, including Wavelet transform, Laplace transform, fast Fourier transform, discrete Fourier transform, short-time Fourier transform, Z-transform and singular value decomposition, as a substitute of Fourier transform.

9. A snoring sound detection method according to claim 3, wherein the method comprises converting a plurality of received signals to a plurality of envelopes of the received signals or to a plurality of a power of envelopes of the received signals, as a plurality of sound intensity signals.

10. A snoring sound detection method according to claim 9, wherein the method comprises calculating a plurality of envelopes of the received signals by one of envelope estimation algorithm including rectification followed by low-pass filtering, magnitude of analytic signal, peak envelope and root-mean-square envelope.

11. A snoring sound detection method according to claim 3, wherein the method comprises converting a plurality of received signals to a plurality of moving average of the absolute values of the received signals or to a plurality of a power of moving average of the absolute values of the received signals, as a plurality of sound intensity signals.

12. A snoring sound detection method according to claim 3, wherein the method comprises evaluating the validity of the periodicity of sound intensity signal by the judgment criteria that a received signal is valid as snoring sound when the periodicity of the sound intensity of the received signal is close to respiratory frequency or its double.

13. A snoring sound detection method according to claim 12, further comprising:
adjusting the possible respiratory frequency according to subject information.

14. A snoring sound detection method according to claim 5, wherein the method comprises evaluating the validity of the periodicity of sound intensity signal by the judgment criteria that a received signal is valid as snoring sound when both the following two conditions are satisfied;
one is that sound intensity signal in the frequency domain after Fourier transform application has one or more local maximums within the frequency band from 0.1 to 5 Hz;
the other is that frequency of the maximum among local maximums within the frequency band from 0.1 to 5 Hz ranges from 0.15 to 2 Hz.

15. A sleep-disordered breathing estimation method according to claim 4, wherein the method comprises estimating apnea-hypopnea index as an index that estimates the prevalence of sleep-disordered breathing;
the apnea-hypopnea index is estimated using the sum of snoring duration per hour normalized by a snoring duration unit;
the snoring duration unit ranges from 20 to 40 s; and
the snoring duration is calculated by the duration of received signals detected as snoring sound.

16. A sleep-disordered breathing estimation method according to claim 4, wherein the method comprises estimating apnea-hypopnea index as an index that estimates the prevalence of sleep-disordered breathing;
the validity of the periodicity of sound intensity signal in terms of respiratory rate is evaluated;
the duration of sound intensity signal ranges from 20 to 40 s; and
the apnea-hypopnea index is estimated using the number of sound intensity signals per one hour judged valid.

17. A snoring sound detection method according to claim 3, wherein received signals of low intensity are excluded from analysis.

18. A snoring sound detection method according to claim 3, further comprising:
excluding received signals converted from sounds acquired during a certain time after sleep and/or a certain time before waking from analysis.

19. A snoring sound detection method according to claim 3, further comprising:
excluding received signals converted from sounds acquired during a subject is awake including speaking.

20. A snoring sound detection method according to claim 3, further comprising: excluding received signals converted from sounds acquired during a subject is supposed to have REM sleep.

21. A sleep-disordered breathing estimation method according to claim 15, wherein the method comprises decreasing the sum of snoring duration when snoring continues for a certain period in the calculation of the sum of snoring duration.

22. A snoring sound detection method according to claim 14, further comprising:
evaluating the validity of the periodicity of sound intensity signal by the judgment criteria that a received signal is valid as snoring sound when the maximum among local maximums within the frequency band from 0.15 to 2 Hz is sufficiently large compared with the intensity of surrounding frequencies.

23. A snoring sound detection method according to claim 22, further comprising:
evaluating the validity of the periodicity of sound intensity signal by the judgment criteria that a received signal is valid as snoring sound when the maximum among local maximums within the frequency band from 0.15 to 2 Hz is large compared with the intensity of surrounding frequencies and the frequency of the maximum is close to one or plural frequencies of the maximums of nearby received signals.

24. A sleep-disordered breathing estimation method according to claim 14, wherein the method increases the estimation value of apnea-hypopnea index when the frequency of the maximum among local maximums within the frequency band from 0.15 to 2 Hz varies largely in a sleep.

25. A snoring sound detection apparatus, comprising:
one or plural microphones that receive a plurality of sounds produced by a subject; and
a controller comprising circuitry configured to convert the sounds produced by the subject to a plurality of received signals, estimate fundamental frequency of each of the received signals, apply a high-pass filter to the received signals, calculate envelopes of high-pass filtered received signals; evaluate periodicity of the envelopes of the high-pass filtered received signals in terms of fundamental frequencies of the received signals, and calculate one or plural indices to detect snoring.

26. A snoring sound detection method, comprising:
acquiring a plurality of sounds produced by a subject;
converting the sounds produced by the subject to a plurality of received signals;
storing the received signals and/or filtered received signals;
estimating fundamental frequencies of the received signals;
applying a high-pass filter to the received signals;
calculating envelopes of high-pass filtered received signals
estimating periodicity of the envelopes of the high-pass filtered received signals;
evaluating the periodicity of the envelopes of the high-pass filtered received signals in terms of fundamental frequencies of the received signals; and
calculating one or plural indices to detect snoring.

27. A snoring sound detection method according to claim 26, wherein the method comprises applying Fourier transform to received signals; calculating intensity of received signals in the frequency domain; and searching fundamental frequencies of received signals;
the duration of the time window for Fourier transform is 1 s or less, and
one of window functions, including rectangular window, B-spline window, Hann window, Hamming window, and Tukey window, is applied to received signals before Fourier transform.

28. A snoring sound detection method according to claim 27, further comprising:
adding data without information to the beginning or/and end of each received signal.

29. A snoring sound detection method according to claim 27, further comprising:
applying interpolation to intensity of received signals in the frequency domain after application of Fourier transform.

30. A snoring sound detection method according to claim 27, wherein the method comprises to employ one of Fourier-related transforms, including Wavelet transform, Laplace transform, fast Fourier transform, discrete Fourier transform, short-time Fourier transform, Z-transform and singular value decomposition, as a substitute of Fourier transform.

31. A snoring sound detection method according to claim 26, wherein the method comprises to apply a high-pass filter with a cutoff frequency to received signal;
the cutoff frequency of a high-pass filter is higher than the fundamental frequency of the received signal.

32. A snoring sound detection method according to claim 26, further comprising:
storing indices;
using the stored indices for the calculation of one or plural indices to detect snoring.

33. A snoring sound detection method according to claim 26, wherein the method comprises detecting the local maxima of the intensity of received signal in the frequency domain; and determining the local maximum with the lowest frequency as the fundamental frequency.

34. A snoring sound detection method according to claim 33, wherein the method comprises determining one of the local maxima of the intensity of received signal in the frequency domain as the fundamental frequency by judging criteria using the amplitude of each local maximum, the intensity of each local maximum, the distance of each local maximum to other local maxima in the frequency domain, and the prominence of each local maximum.

35. A snoring sound detection method according to claim 26, wherein the method comprises estimating the fundamental frequency of each received signal by calculating the periodicity of the signal amplitude in time domain.

36. A snoring sound detection method according to claim 26, wherein the method comprises determining the fundamental frequency of received signals using a plurality of conditions including the fundamental frequencies are in the range from 10 to 300 Hz.

37. A snoring sound detection method according to claim 26, wherein the method comprises calculating the envelope of high-pass filtered received signals under the condition of the envelope frequency being in the range from 10 to 300 Hz.

38. A snoring sound detection method according to claim 26, wherein the method comprises applying Fourier transform to the envelopes of high-pass filtered received signals; and searching the maximum of local maximum of the intensity of each received signal in the frequency domain after Fourier transform in order to estimates periodicity of the envelopes of high-pass filtered received signals.

39. A snoring sound detection method, comprising:
acquiring a plurality of sounds produced by a subject;
converting the sounds produced by the subject to a plurality of received signals;
storing the received signals and/or filtered received signals;
estimating fundamental frequencies of the received signals;
searching second harmonics of the received signals,
applying a high-pass filter to the received signals;
calculating envelopes of high-pass filtered received signals
estimating periodicity of the envelopes of the high-pass filtered received signals;
evaluating the periodicity of the envelopes of the high-pass filtered received signals in terms of fundamental frequencies of the received signals; and
calculating one or plural indices to detect snoring.

40. A snoring sound detection method according to claim 39, wherein the method comprises searching the maximum of local maxima of the intensity of each received signal in the frequency range from the fundamental frequency to the second harmonic of the received signal; and judging that the received signal may include snoring sound when both the intensity of fundamental frequency and the intensity of the second harmonic of the received signal are higher than the maximum of local maxima of the intensity of each received signal in the frequency range from the fundamental frequency to the second harmonic of the received signal.

41. A snoring sound detection method according to claim 39, wherein the method comprises searching the maximum of the intensity of each received signal in the frequency range less than the fundamental frequency of the received signal; and judging that the received signal may include snoring sound when the intensity of fundamental frequency of the received signal is larger than the maximum of the intensity of each received signal in the frequency range less than the fundamental frequency of the received signal.

42. A snoring sound detection method, comprising:
acquiring a plurality of sounds produced by a subject;
converting the sounds produced by the subject to a plurality of received signals;
storing the received signals and/or filtered received signals;
applying Fourier transform to the received signals;
calculating intensity of the received signals in a frequency domain;
searching fundamental frequencies of the received signals;
investigating whether low frequency components of the received signals are dominant;
applying a high-pass filter to the received signals;
calculating envelopes of high-pass filtered received signals
estimating periodicity of the envelopes of the high-pass filtered received signals;
evaluating the periodicity of the envelopes of the high-pass filtered received signals in terms of fundamental frequencies of the received signals; and
calculating one or plural indices to detect snoring.

43. A snoring sound detection method according to claim 42, wherein the method comprises judging that the received signal may include snoring sound when the intensity of the fundamental frequency of received signal is dominant in the frequency domain.

44. A snoring sound detection method according to claim 42, wherein the method comprises judging that the received signal may include snoring sound when the intensity of the fundamental frequency of received signal accounts for 10% or more of the intensity sum of the received signal.

45. A snoring sound detection method according to claim 42, wherein the method comprises judging that the received signal may include snoring sound when the intensity and/or prominence of the fundamental frequency is higher than the intensity and prominence of other frequencies.

46. A snoring sound detection method, comprising:
acquiring a plurality of sounds produced by a subject;
converting the sounds produced by the subject to a plurality of received signals;
storing the received signals and/or filtered received signals;
applying a high-pass filter with a cutoff frequency of 20 Hz or less to the received signals;
calculating autocorrelation coefficients of the received signals in a time domain using sliding windows of plural window widths; and
judging that the received signals include snoring sound when autocorrelation coefficients of the received signals become maximum in case of employing a window width of 20 ms or more,
wherein a range of time lag for autocorrelation-coefficient calculation is included in a range from half the window width to twice the window width.

47. A snoring sound detection method according to claim 3, further comprising:
storing a plurality of received signals and/or filtered received signals.
